(19) United States
(12) Patent Application Publication
(10) Pub. No.: US 2002/0038078 A1
(43) Pub. Date: Mar. 28, 2002

(54) Apparatus for measuring/determining concentrations of light absorbing materials in blood

(75) Inventor: Kazumasa Ito, Tokyo (JP)

Correspondence Address: SUGHRUE, MION, ZINN, MACPEAK & SEAS, PLLC, 2100 Pennsylvania Avenue, N.W.,, Washington, DC, 20037, US

(73) Assignee: NIHON KOHDEN CORPORATION

(21) Appl. No.: 09/960,740

(22) Filed: Sep. 24, 2001

(30) Foreign Application Priority Data
Sep. 22, 2000 (JP) P2000-287973

(51) Int. Cl. A61B 5/00

(52) U.S. Cl. 600/309; 600/323; 600/324

(57) ABSTRACT

An apparatus for measuring/determining concentrations of light absorbing materials in blood includes a probe 10 and a measurement device main body 30. A plurality of optical signals of different wavelengths are irradiated onto living tissue, and the concentrations of light absorbing materials in blood is determined by means of a photoplethysmogram detected by means of the light that has transmitted through the tissue. Simulating-signal generating means is provided in the measurement device main body and generates an arbitrary simulating-pulse-wave signal corresponding to the photoplethysmogram detected by the probe.

### BACKGROUND OF INVENTION

1. Field of invention

The present invention relates to an apparatus for measuring/determining concentrations of light absorbing materials in blood of living tissue, the apparatus having a measuring device for computing the concentrations of light absorbing materials in living tissue. Specifically, the present invention relates to a check-up system for use with an apparatus for measuring/determining concentrations of light absorbing materials in blood. Particularly, the present invention relates to an apparatus for measuring/determining concentrations of light absorbing materials in blood, having a self checkup function for checking whether or not the apparatus and a probe function normally. The probe is brought into close proximity to or into contact with living tissue.

2. Related art

A pulse oximeter capable of measuring oxygen saturation in arterial blood has already been known as an apparatus for measuring/determining the concentrations of light absorbing materials in living tissue. The pulse oximeter is known as a device which measures consecutively and non-invasively an oxygen saturation in arterial blood (SpO₂), by utilization of variations in the amount of blood flowing through the artery caused by a stroke.

The pulse oximeter enables extraction of only information about arterial blood by use of photoplethysmogram. Light is irradiated onto comparatively-thin living tissue, such as a finger, and the intensity of the light that has passed through the tissue (i.e., photoplethysmogram) is recorded. More specifically, the light absorbing characteristic of blood changes according to an oxygen saturation level. Accordingly, even in the case of pulsation in which the same amount of blood fluctuates, a resultant pulse wave amplitude varies according to the oxygen saturation of the blood.

As shown in FIG. 8, the pulse oximeter generally comprises a probe 10 to be attached to a patient, and a measurement device main body 20. The probe 10 is provided with a light-emitting section 12 and a light-receiving section 14. The light-emitting section 12 and the light-receiving section 14 are arranged such that a measuring site (i.e., living tissue), such as a finger 16, is placed between the light-emitting section 12 and the light-receiving section 14. Two light-emitting diodes (LED1 and LED2); namely, one having a light-emitting wavelength of 660 nm (red light) and the other having a light-emitting wavelength of 940 nm (infrared light), are employed for the light-emitting section 12. Further, a photodiode PD is employed for the light-receiving section 14.

By way of a light-emitting diode drive circuit 23, the two light-emitting diodes LED1 and LED2 alternately emit at predetermined timings set by a timing generation circuit 22 provided in the measurement device main body 20.

The light-emitting diode LED 1 and the light-emitting diode LED 2 of the light-emitting section 12 alternately output light. The intensity of light of respective wavelengths (660 nm and 940 nm) that has passed through tissue of the finger 16 or the like and arrived at the light-receiving section 14 is converted into an electric current by means of the photodiode PD. A current-to-voltage converter 24 provided in the measurement device main body 20 converts the electric current into a voltage. A demodulator 25 separates the voltage into transmitted-light signals of the two wavelengths.

From the two transmitted-light signals produced by the demodulator 25, a pulse-wave component detector 26a (detecting light absorbance at a wavelength of 660 nm) extracts a pulse-wave component of absorbance (ΔA660). Similarly, a pulse-wave component detector 26b (detecting light absorbance at a wavelength of 940 nm) extracts a pulse wave component of absorbance (ΔA940). A light absorbance ratio calculator 27 calculates a ratio of absorbance Φ=ΔA660/ΔA940. An oxygen saturation converter 28 converts the absorbance ratio into oxygen saturation S=f(φ).

The apparatus of pulse spectro photometry type for measuring/determining concentrations of light absorbing materials in blood; e.g., a pulse oximeter, can perform consecutive and non-invasive measurement and in theory needs no calibration for each measurement. The apparatus satisfies basic demands for monitoring the condition of a patient. Hence, the apparatus has been conventionally adopted and become widespread as a vital sign monitor.

However, when the apparatus having the foregoing configuration is used as a vital sign monitor, to check whether or not the monitor is operating appropriately is important and inevitable for a patient's safety.

In light of this, there have already been proposed a checkup system and a test device for checking the concentration determination apparatus. The system and device can check whether or not a probe and a measurement device main body operate normally and effectively in terms of safety and reliability. There have also been proposed a checkup system and a test device, which have been constructed so as to be able to perform checkup for reliable operation of the apparatus.

A related-art checkup system comprises a probe 10 and a checkup device. In order to check appropriate operation of the measurement device main body, the probe is separated from the measurement device main body. There is provided a checkup device capable of outputting a preset checkup signal (reference value) corresponding to a vital sign acquired through the probe. The checkup device is connected to the measurement device main body, thereby enabling checking of whether or not the measurement device main body operates normally. So long as the probe that has been separated from the measurement device main body is connected to the checkup device, the checkup device can check the sensitivity of a sensor for detecting variations in a vital sign of the probe.

The related-art test device is provided with a tissue model or a blood model. The tissue model or the blood model is made so that a light absorbance characteristic approximating pulsation of blood in living tissue can be realized artificially. The measurement device main body is subjected to testing using the model.

The checkup system provided in the previously-described related-art apparatus is provided with a checkup device having a special function. When the checkup device is in use, the probe is separated from the measurement device main body. The measurement device main body and the probe are individually connected to the checkup device. As a result, the measurement device main body can be checked for normal operation, and the sensitivity of the probe can be checked separately. Accordingly, a problem of such a checkup system is taking a lot of time and trouble.

The related-art test apparatus encounter a problem of a complicated configuration of the apparatus including a tissue model or a blood model with increasing manufacturing costs.

As mentioned previously, in an apparatus, such as a pulse oximeter, for measuring/determining concentrations of light absorbing materials in blood in view of a photoplethysmogram detected by irradiating a plurality of optical signals in different wavelengths onto living tissue and passing therethrough, a light-emitting diode (LED) is used as a probe for detecting photoplethysmogram. Even though the amount of light to be emitted from the LED can be controlled by supplying the electric current to the LED, it is difficult to accomplish the required accuracy, e.g., in the checkup of a pulse oximeter. FIG. 9 shows an example of relationship between electric current (mA) to be supplied to a red LED and a current (μA) received by a photodiode (PD) (i.e., the intensity of light received by a probe), as well as an example of relationship between electric current (mA) to be supplied to an infrared LED and the current received by the photodiode. From FIG. 9, it is understood that the respective relationships are not completely proportional, and that the characteristic of the red LED differs from that of the infrared LED. Even in the case of LEDs of identical color, each characteristic of LED cannot be limited, because of variations. For these reasons, in relation to the apparatus for measuring and determining concentrations of light absorbing materials in blood when a probe is replaced frequently, integrating a probe and a checkup function of a measurement device main body is considerably difficult.

### SUMMARY OF INVENTION

The present inventor has conceived simulating-signal generating means for use in an apparatus, which determines concentrations of light absorbing materials in blood, comprising a probe and a measurement device main body. The simulating-signal generating means generates an arbitrary simulating-pulse-wave signal corresponding to a photoplethysmogram which has been detected in the apparatus by the probe. In the apparatus, a plurality of optical signals of different wavelengths are irradiated to living tissue and transmitted through the living tissue to detect the photoplethysmogram by the probe, and main body determines concentrations of light absorbing materials in blood. The measurement device main body measures concentrations of light absorbing materials in blood.

The present inventor has ascertained the following: The simulating-signal generating means enables easy and quick checkup of an appropriate state of the probe. A control signal to be used for irradiating optical signals is configured so as to bypass the probe in the measurement device main body by way of signal switching means which selectively switches a signal output from the probe and the simulating-pulse-wave signal. Thus, the present inventor has found that there can be provided an apparatus for measuring/determining concentrations of light absorbing materials in blood having a self checkup function capable of readily checking whether or not a measurement device main body operates normally, through use of a comparatively simple configuration and without detaching the probe from the device main body.

The present invention is aimed at providing an apparatus for measuring/determining the concentrations of light absorbing materials in blood, the apparatus having a self checkup function capable of readily checking whether or not a measurement device main body operates normally, through use of a comparatively simple configuration and without detaching the probe from the device main body as well as capable of readily and quickly checking the probe as to an appropriate state thereof.

To achieve the object, the present invention provides an apparatus for measuring/determining concentrations of light absorbing materials in blood comprising:

a probe for detecting a photoplethysmogram by irradiating and passing a plurality of optical signals of different wavelengths onto and through a living tissue;

a measurement device main body for determining concentrations of light absorbing materials in blood on the basis of the pulse spectrophotometry;

simulating-signal generating means, for generating an arbitrary simulating-pulse-wave signal corresponding to the photoplethysmogram detected by the probe, provided in the measurement device main body

According to the apparatus of the present invention, the probe irradiates the optical signals in the probe on the basis of the simulating pulse wave signal to detect a simulating-pulse-wave.

The apparatus for measuring/determining concentrations of light absorbing materials in blood further comprises a self checkup function based on a result of processing of the simulating-pulse-wave in the device main body.

The present invention provides the apparatus for measuring/determining concentrations of light absorbing materials in blood, further comprising:

self checkup simulating-signal generating means, for generating an arbitrary simulating-pulse-wave signal corresponding to the photoplethysmogram detected by a probe, provided in the measurement device main body

a bypass interconnection, arranged in the measurement device main body, routed so as to bypass a probe to be adapted; and

signal switching means for selectively inputting the photoplethysmogram detected by the probe and the simulating-pulse-wave signal which is transmitted through the bypass interconnection into a signal input section of the measurement device main body.

Here, the simulating-pulse-wave signal produced by the simulating signal generating means is transmitted such that the signal switching means selectively inputs, to the signal input section, a signal transmitted by way of a bypass interconnection and a received-light signal which is detected as a result of the optical signals having been irradiated in the probe corresponding to the simulating pulse-wave signal, thereby identifying an anomalous condition of the measurement device main body as a self checkup function.

Further, the simulating-pulse-wave signal produced by the simulating signal generating means is transmitted such that the signal switching means selectively inputs, to the signal input section, a signal transmitted by way of the bypass interconnection and a received-light signal which is detected as a result of the optical signals having been irradiated in the probe corresponding to the simulating pulse-wave signal, thereby identifying a normal operating state of the apparatus, an anomalous condition of the probe, and an anomalous condition of the measurement device main body, as a self checkup function.

Preferably, the apparatus for measuring/determining concentrations of light absorbing materials in blood further comprises a display section for displaying a checkup status of the apparatus, a normal operating status of the apparatus, an anomalous condition of the probe, and an anomalous condition of the measurement device main body.

Preferably, the bypass interconnection is provided with signal conversion means for converting the simulating-pulse-wave signal into a photoplethysmogram signal detected by the probe.

Preferably, the simulating signal generating means controls, through pulse-width modulation (PWM) control, a time during which the respective light-emitting diodes are emitting in accordance with simulating-pulse-wave signal, thereby producing a required simulating-pulse-wave received-light signal.

Preferably, the simulating signal generating means controls in accordance with the simulating-pulse-wave signal, through pulse width modulation (PWM) control, an extraction time required for demodulating a received-light signal stemming from emitting of respective light-emitting diodes being irradiated, thereby producing a required simulating-pulse-wave received-light signal.

Preferably, the shape of a simulating pulse wave is set in accordance with a pulse-width modulation (PWM) pattern and in connection with a relationship between the pulse-width modulation (PWM) and a simulating-pulse-wave received-light signal.

Preferably, a pulsation component rate (the ratio between an AC component and a DC component) of a simulating pulse wave is set in accordance with a pulse-width modulation (PWM) ratio and in connection with a relationship between the pulse-width modulation (PWM) and a simulating-pulse-wave received-light signal.

Preferably, a simulating pulse rate is set in accordance with a modulation cycle and in connection with a relationship between the pulse-width modulation (PWM) and a simulating-pulse-wave received-light signal.

Preferably, a parameter pertaining to an absorption coefficient rate is set on the basis of a modulation ratio proportion between wavelengths and in connection with a relationship between the pulse-width modulation (PWM) and a simulating-pulse-wave received-light signal.

Preferably, the simulating-pulse-wave signal is formed from integral values (i.e., an integral value means an area) obtained by integrating separated light-receiving time by a demodulation circuit section to each wavelength.

### BRIEF DESCRIPTION OF DRAWINGS

FIG. 1 is a block diagram showing the circuitry of a pulse oximeter having a self checkup function, the oximeter being an embodiment of an apparatus for measuring/determining concentrations of light absorbing materials in blood according to the present invention;

FIG. 2 is a descriptive view showing generation of a photoplethysmogram produced by a probe of the pulse oximeter shown in FIG. 1, a waveform characteristic of a received-light signal, and a waveform characteristic of a demodulation signal;

FIG. 3 is a descriptive view showing generation of a simulating-pulse-wave signal performed by simulating signal generating means of the pulse oximeter shown in FIG. 1, a waveform characteristic showing one embodiment of the received-light signal, and a waveform characteristic showing one embodiment of the demodulation signal;

FIG. 4 is a descriptive view showing generation of a simulating-pulse-wave signal performed by simulating signal generating means of the pulse oximeter shown in FIG. 1, a waveform characteristic showing another embodiment of the received-light signal, and a waveform characteristic showing another embodiment of the demodulation signal;

FIG. 5 is a descriptive view showing an example of checkup mode selection display screen appearing on a display screen of a measurement device main body of the pulse oximeter shown in FIG. 1;

FIG. 6 is a descriptive view showing an example of checkup status display appearing on a display screen of a measurement device main body of the pulse oximeter shown in FIG. 1;

FIG. 7 is a flowchart for a control program which enables automatic checkup of the measurement device main body of the pulse oximeter shown in FIG. 1;

FIG. 8 is a block diagram showing the circuitry of a related-art pulse oximeter; and

FIG. 9 is a plot showing the relationship between received-light current of a PD and electric current used for a plurality of LEDs having different wavelengths.

### DETAILED DESCRIPTION OF PREFERRED EMBODIMENTS

Embodiments of an apparatus for measuring/determining concentrations of light absorbing materials in blood having a self checkup function on the basis of pulse spectrophotometry according to the present invention will be described in detail referring to the accompanying drawings.

First Embodiment

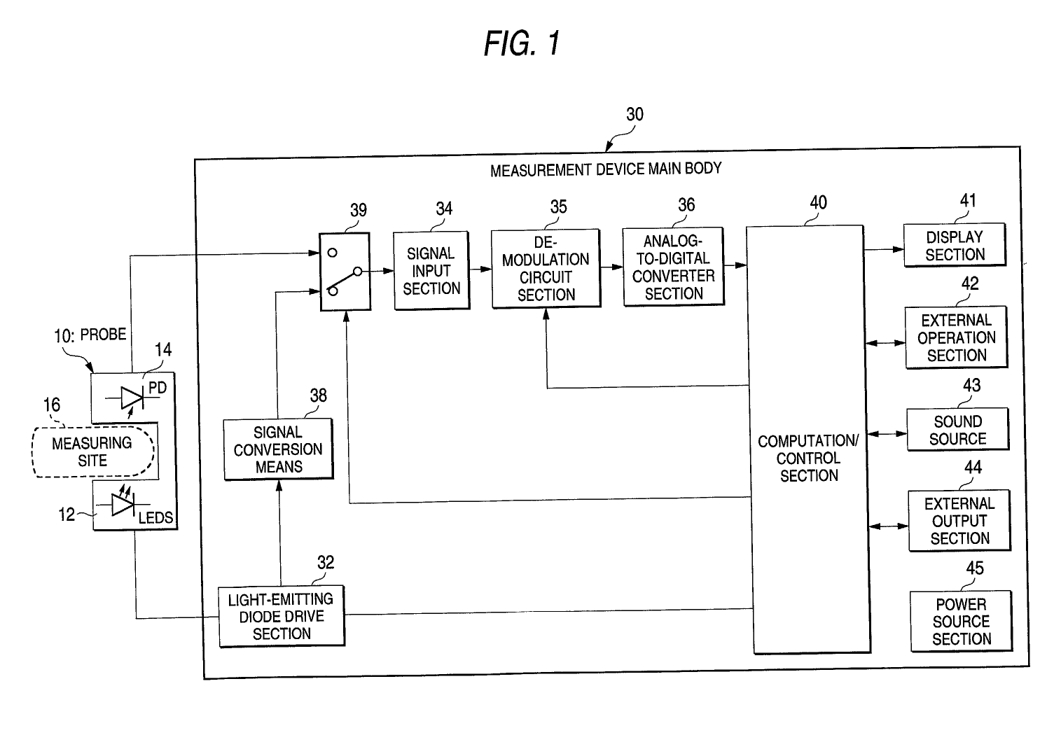

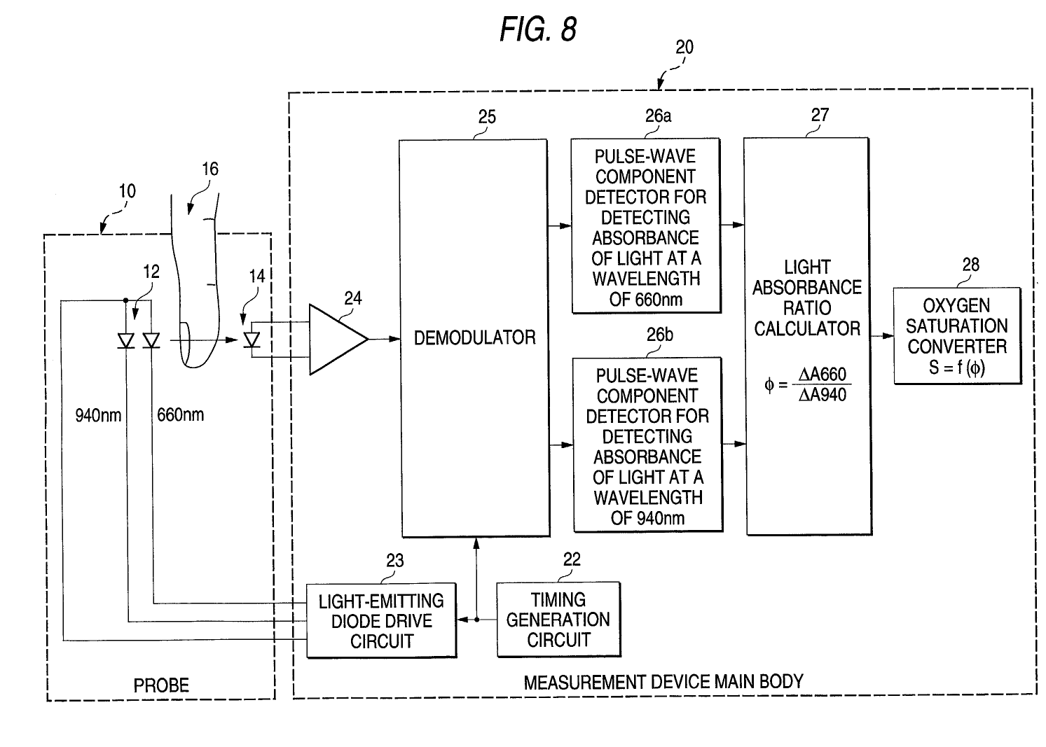

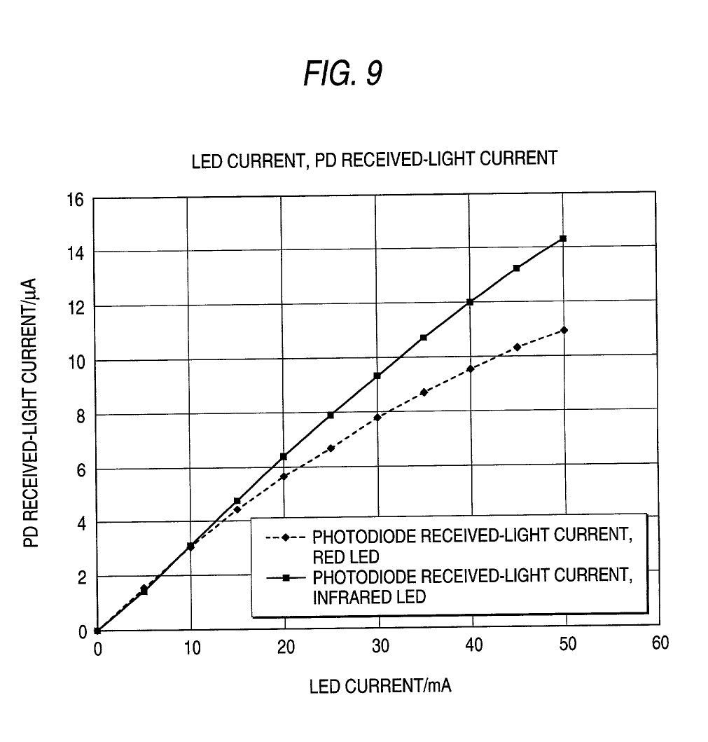

FIGS. 1 through 3 show an embodiment of an apparatus for measuring/determining concentrations of light absorbing materials in blood having a self checkup function according to the present invention. For convenience in explanation, constituent elements which are identical with those of the related-art pulse oximeter shown in FIG. 8 will be described using the reference numerals.

As shown in FIG. 1, the pulse oximeter according to the present embodiment comprises a probe 10 for detecting a photoplethysmogram in living tissue (e.g., a finger) 16 serving as a measuring site, and a measurement device main body 30 for measuring/determining concentrations of light absorbing materials in blood of the living tissue 16 on the basis of the photoplethysmogram.

As in the case of the related-art pulse oximeter, the probe 10 is provided with a light-emitting section 12 consisting of a plurality of light-emitting diodes LEDs, and a light-receiving section 14 using a photodiode PD. The light-emitting section 12 and the light-receiving section 14 are arranged such that the measuring site (living tissue) 16 is interposed between the light-emitting section 12 and the light-receiving section 14. A light-emitting diode R-LED having a light-emitting wavelength of 660 nm (red R) and a light-emitting diode IR-LED having a light-emitting wavelength of 940 nm (infrared IR) are used for the light-emitting section 12.

The measurement device main body 30 is provided with a light-emitting diode drive section 32 for alternately emitting light-emitting diodes LEDs in the light-emitting section 12 of the probe 10. Further, the main body 30 is provided with a signal input section 34 for entering a signal (current) obtained by a photodiode (PD) provided in the light-receiving section 14. As in the case of the related-art pulse oximeter, the signal input to the signal input section 34 is demodulated by a demodulation circuit section 35. The thus-modulated signal is transferred through an analog-to-digital converter section 36, to a computation/control section 40 which converts the signal into a required measurement value or performs required control operation.

In relation to the measurement device main body 30 of the present embodiment, simulating signal generating means is provided in the computation/control section 40. A simulating-pulse wave signal produced by the simulating signal generating means 39 and a signal output from the photodiode PD of the probe 10 can be selectively input to the signal input section 34 by signal switching means 39, byway of the probe 10 connected between the light-emitting diode drive section 32 and the signal switching means as well as by way of signal conversion means 38 interconnected so as to bypass the probe 10. The present invention is not limited by this embodiment. It is applicable for equipping the simulating signal generating means outside the computation/control 40, by which the simulating signal generating means is controlled.

In this case, the computation/control section 40 is set so as to output a control signal to be used for causing the signal switching means 39 to perform switching operation. Further, the computation/control section 40 is set so as to subject, to modulation control, either a light-emitting control signal to be sent to the light-emitting diode drive section 32 or a demodulation control signal produced by the demodulation circuit section 35, for the purpose of producing a simulating signal.

The computation/control section 40 is connected to a display section 41, an external operation section 42, a sound source 43, and an external output section 44 and is arranged so as to perform required operation and control. The measurement device main body 30 is provided with a power source section 45 for causing the main body 30 and the probe 10 to perform electrical operation, as required.

The operation of the pulse oximeter having the foregoing configuration according to the present embodiment will now be described.

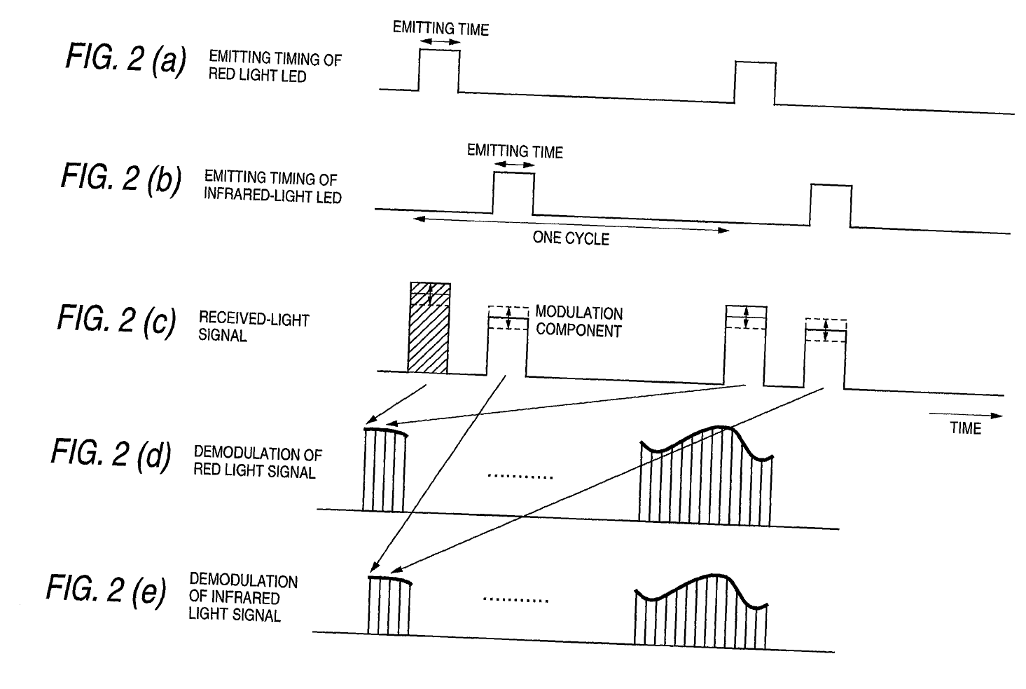

When the concentrations of light absorbing materials in blood of the living tissue 16 is measured usually, the probe 10 is connected to the measurement device main body 30. The computation/control section 40 produces a timing at which the light-emitting diode R-LED of the light-emitting section 12 is to emit light [see FIG. 2(a)] and a timing at which the light-emitting diode IR-LED of the same is to emit light [see FIG. 2(b)]. The light-emitting diode drive section 32 causes the respective light-emitting diodes R-LED and IR-LED to emit. The light emitted from the light-emitting diode R-LED and the light-emitting diode IR-LED reaches the photodiode PD of the light-receiving section 14 after having passed through the measuring site (i.e., living tissue) 16.

As mentioned above, the photo/electric converted signal (electric current) converted by the photodiode PD is input to the signal input section 34 by way of the signal switching means 39, where the electric current is converted into a voltage. Accordingly, a component—on which an optical characteristic of pulsating action at the measuring site 16 is reflected—appears, as a modulation component of amplitude, on a received-light signal obtained by the signal input section 34 as shown FIG. 2(c). The demodulation circuit section 35 separates the signals received by the light-emitting diodes R-LED and IR-LED from each other and demodulates the thus-separated signal as shown in FIGS. 2(d) and 2(e), thereby obtaining a signal required for computing a SpO₂ value (arterial oxygen saturation).

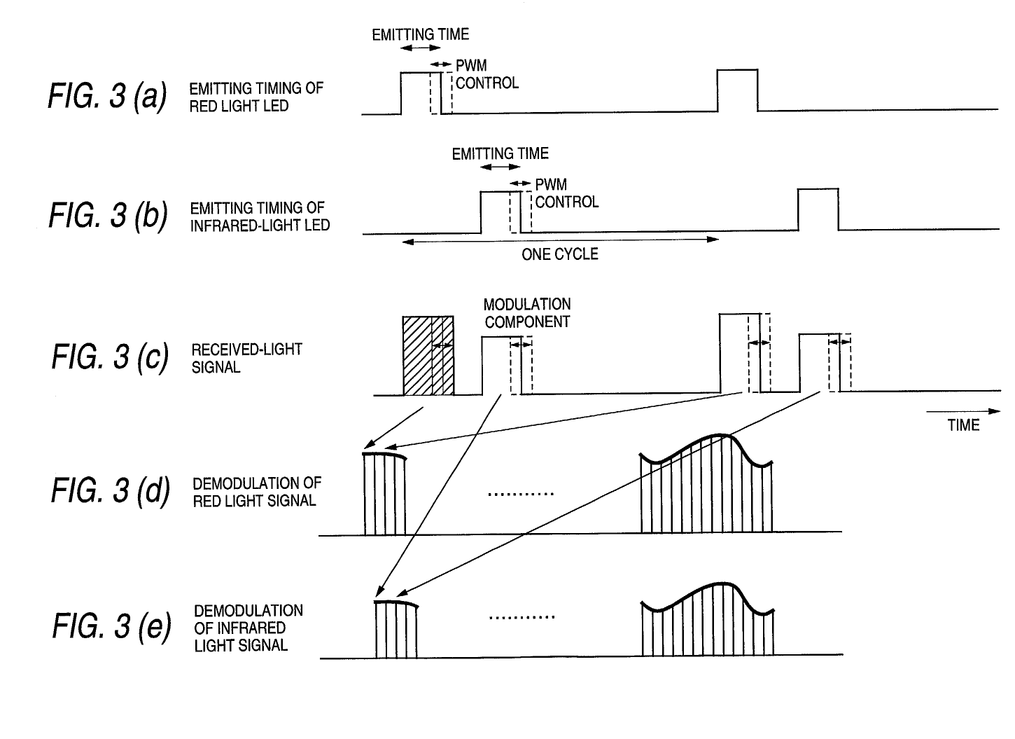

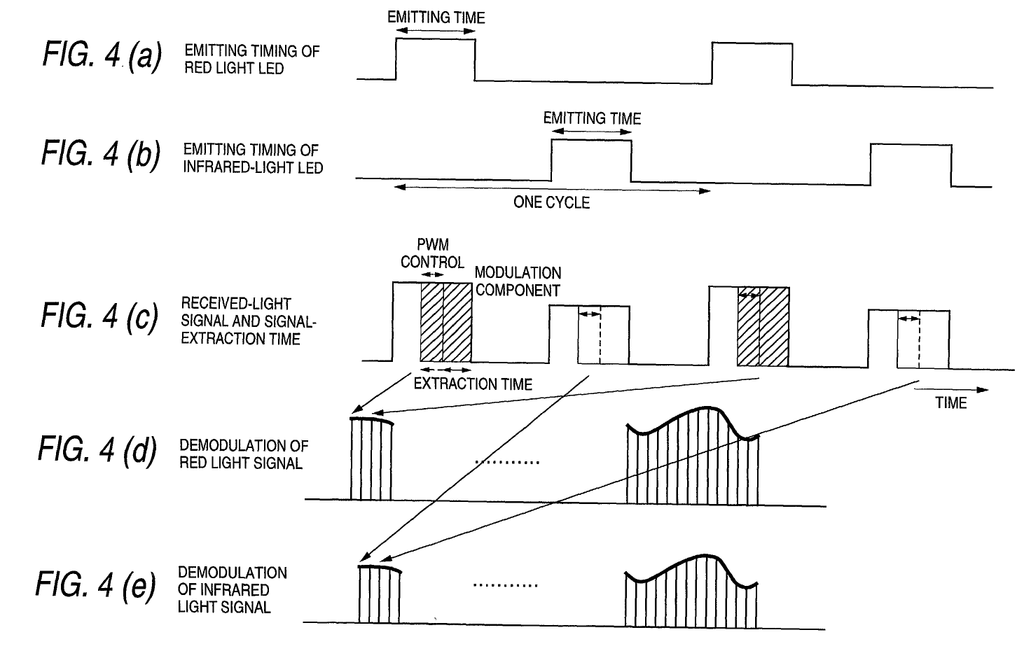

In checking the operation of the pulse oximeter according to the present embodiment, a modulation component of amplitude—on which pulsating action is reflected—is obtained from the measuring site 16 on the probe 10. In place of the modulation component of amplitude, checkup is realized by use of a simulating-pulse-wave signal produced by the simulating signal generating means provided in the computation/control section 40 of the measurement device main body 30. In this case, a component corresponding to the modulation component of amplitude can be embodied, by means of subjecting, to pulse-width modulation (PWM) control, the time during which the light-emitting diode R-LED is emitting and the time during which the light-emitting diode IR-LED is emitting as FIGS. 3(a) through 3(e). Alternatively, when a portion of a received-light signal is extracted within-a period of a light-emitting timing and the thus-extracted signal is demodulated, checkup can be implemented by means of subjecting, to pulse-width modulation (PWM) control, the extraction time required for demodulating the received-light signal as shown in FIGS. 4(a) through 4(e) The method of producing a simulating photoplethysmogram can be implemented by a pulse oximeter, wherein the demodulation circuit section 35 separates the light-receiving times from each other and obtain a pulse wave component from an integral value (area) of each of the received-light signals.

In the present embodiment, the time during which the light-emitting diode R-LED is emitting in accordance with a simulating-pulse-wave signal and the time during which the light-emitting diode IR-LED is emitting in accordance with the same are subjected to pulse-width modulation (PWM) control, thereby enabling the computation/control section 40 to produce a required simulating pulse wave received-light signal. In this case, the following relationship stand between pulse width modulation (PWM) and a simulating pulse wave received-light signal.

(1) The shape of a simulating pulse wave is set by means of a pulse-width modulation (PWM) pattern.

(2) A pulsation component rate (the ratio between an AC component and a DC component) of a simulating pulse wave is set by means of a pulse-width modulation (PWM) ratio.

(3) A simulating pulse rate is set by means of a modulation cycle.

(4) Parameters (SpO₂ or the like) pertaining to a light absorbance coefficient ratio are set on the basis of a modulation ratio proportion between wavelengths.

The light-emitting time or the time during which a received-light signal is to be extracted during demodulation is set by means of pulse-width modulation (PWM), as required. There can be produced an arbitrary simulating pulse wave signal from an arbitrary wave form. From the arbitrary simulating pulse wave signal an arbitrary amplitude, an arbitrary SpO₂ value, and an arbitrary pulse rate can be obtained.

The thus-produced simulating pulse wave signal can be utilized for checking the measurement device main body 30 and the entire measurement system including the probe 10, through switching action performed by the signal switching means 39. Further, a set value of the simulating pulse wave signal and a result of signal processing can be compared with each other in the measurement device main body 30. The computation/control section 40 can perform automatic checkup operation. The checkup results are compared with each other in association with switching action of the signal switching means 39. As a result, an automatic determination can be made as to whether the pulse oximeter is in a normal operating state, an anomalous condition of a probe, and an anomalous condition of a measurement device main body.

At the time of checkup of the pulse oximeter, predetermined material whose attenuation characteristic is known is applied to the measuring site 16 on the probe 10 or the light-emitting section 12 and the light-receiving section 14 are set so as to mutually oppose under no load (i.e., no living tissue is interposed between the light-emitting section 12 and the light-receiving section 14).

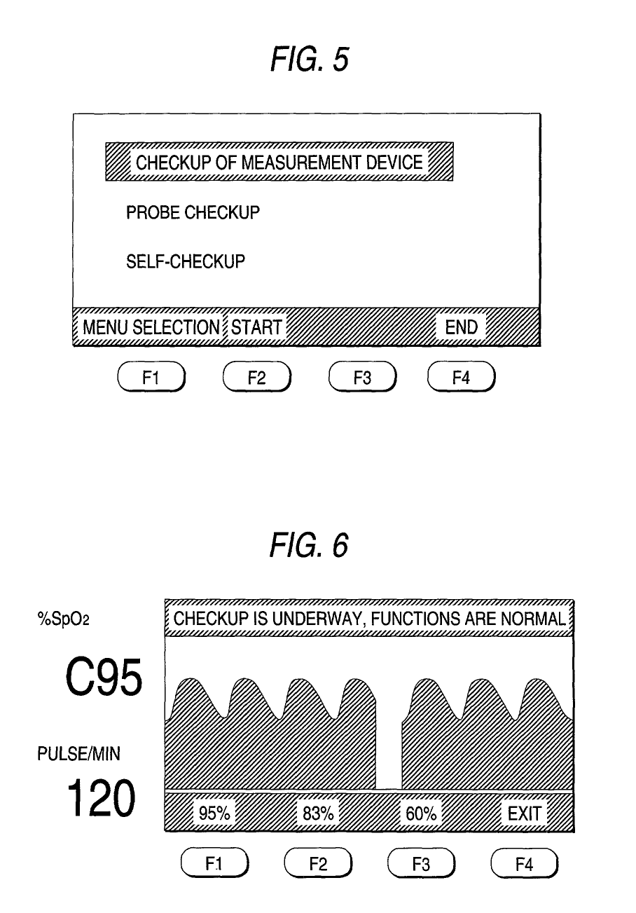

FIGS. 5 and 6 show a display example of the display section 41 provided on the measurement device main body 30 of the pulse oximeter according to the present embodiment. FIG. 6 shows a display example in a mode for checking the measurement device. As shown in FIG. 6, the display section 41 is set to basically the same display function as that employed during an ordinary measurement operation. In this case, there are set an SpO₂ value (“C95” is displayed as “% SPO₂”), a pulse rate (“120” is displayed as “Pulse/min.”), a checkup status (a message stating that “Checkup is underway”: Functions are normal” appears), display of checkup functions (“95%,” “83%,” “60%,” and “exit” are displayed), and selection keys for selecting the display (F1, F2, F3, and F4). “C” in display “C95” pertaining to the value of “SpO₂” indicates a checkup mode so that a user can distinguish the checkup mode from an ordinary measurement mode.

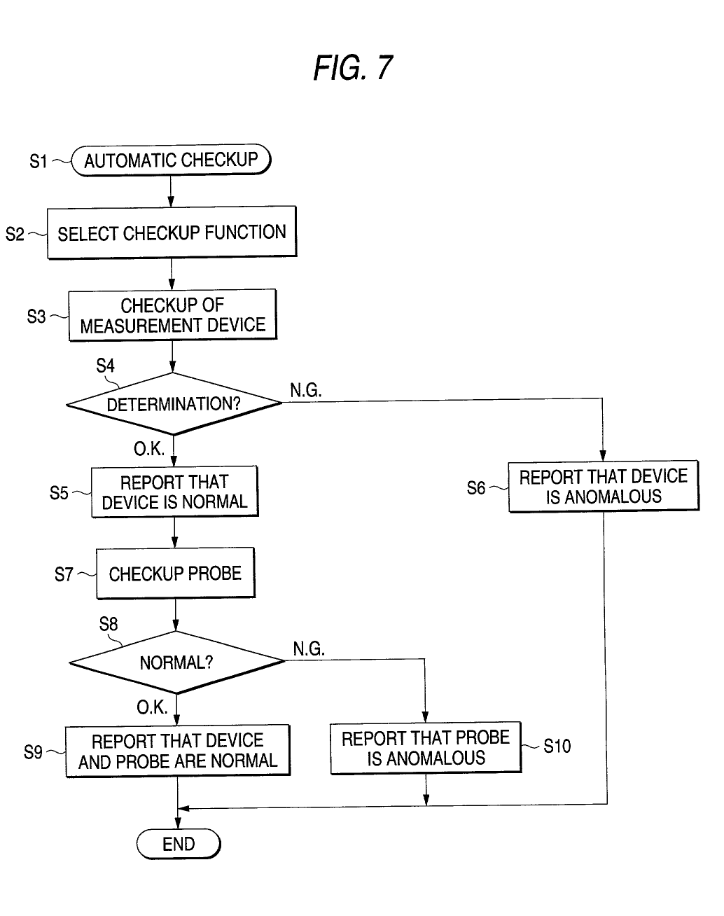

FIG. 7 is a flowchart showing an checkup program employed when automatic checkup of the pulse oximeter is performed. More specifically, in relation to the checkup program shown in FIG. 7, in step S1 automatic checkup is commenced. In step S2, a checkup function is selected for the “measurement device.” In this case, in the pulse oximeter (the measurement device main body 30) a simulating pulse wave signal is produced by the simulating signal generating means provided in the computation/control section 40, and by way of the signal conversion means 38, through a switching operation of the signal switching means 39, is input to the signal input section 34. And the measurement device is checked in step S3.

In step S4, if the measurement device is determined to be normal in accordance with a result of checkup of the measurement device, in step S5 there is reported a notice that the measurement device is normal. For example, as a way to report a notice, a notice that the measurement device is normal is displayed as a checkup status of the display section 41 (see FIG. 6). In contrast, if in step S4 the measurement device is determined to be anomalous, in step S6 a notice that the measurement device is anomalous is reported. Even in this case, the notice can be reported in the same manner. If the measurement device is anomalous, checkup is terminated immediately.

If in step S5 it is reported that the measurement device is normal, in step S7 the probe 10 is checked. In this case, a signal to be detected by the probe is input to the signal input section 34 by means of switching operation of the signal switching means 39, thereby performing checkup of the probe.

If the probe is determined to be normal in step S8 as a result of checkup of the probe, in step S9 it is reported that the measurement device and the probe are normal, and checkup is terminated. If in step S8 the probe is determined to be anomalous, in step S10 it is reported that the probe is anomalous. Checkup of the probe is terminated immediately. Even in this case, the notice is reported in the same manner as mentioned previously.

Although the pulse oximeter has been described as a preferable embodiment, the present invention is not limited to the pulse oximeter. As in the case of the embodiment, the present invention can be applied to an apparatus capable of determining concentrations of light absorbing materials in blood from a photoplethysmogram. As a matter of course, the present invention is susceptible to modifications in design within the scope of the invention.

As is obvious from the foregoing embodiment, the present invention provides an apparatus for measuring/determining concentrations of light absorbing materials in blood comprising a probe and a measurement device main body, wherein a plurality of optical signals of different wavelengths are irradiated onto living tissue, and the concentrations of light absorbing materials in blood are determined by means of a photoplethysmogram detected by means of the light that has transmitted through the tissue; and the apparatus has a probe for detecting a photoplethysmogram and a measurement device main body for measuring the concentrations of light absorbing materials in blood, comprising:

simulating-signal generating means which is provided in the measurement device main body and which generates an arbitrary simulating-pulse-wave signal corresponding to the photoplethysmogram detected by the probe. A checkup can be made simply as to whether or not a measurement device main body operates normally, by means of a simple configuration and without separating the measurement device main body and the probe. A checkup can be made simply and quickly as to an appropriate state of the probe. Thus, the present invention can provide many advantages.

What is claimed is:

1. An apparatus for measuring/determining concentrations of light absorbing materials in blood comprising:
a probe for detecting a photoplethysmogram by irradiating and passing a plurality of optical signals of different wavelengths onto and through a living tissue;
a measurement device main body for determining concentrations of light absorbing materials in blood on the basis of the pulse spectrophotometry;
simulating-signal generating means, for generating an arbitrary simulating-pulse-wave signal corresponding to the photoplethysmogram detected by the probe, provided in the measurement device main body.

2. The apparatus for measuring/determining concentrations of light absorbing materials in blood as claimed in claim 1, wherein the probe irradiate the optical signals in the probe on the basis of the simulating pulse wave signal to detect a simulating-pulse-wave.

3. The apparatus for measuring/determining concentrations of light absorbing materials in blood according to claim 1, further comprising:
means for performing a self checkup function based on a result of processing of the simulating-pulse-wave in the device main body.

4. The apparatus for measuring/determining concentrations of light absorbing materials fin blood according to claim 1, wherein the simulating signal generating means controls a time period for irradiating respective light-emitting diodes in accordance with the simulating-pulse-wave signal through pulse-width modulation (PWM) control to obtain a required simulating-pulse-wave received-light signal.

5. The apparatus for measuring/determining concentrations of light absorbing materials in blood according to claim 1, wherein the simulating signal generating means controls an extraction time required for demodulating a received-light signal stemming from emitting of respective light-emitting diodes being irradiated in accordance with the simulating-pulse-wave signal through pulse width modulation (PWM) control to produce a required simulating-pulse-wave received-light signal.

6. An apparatus for measuring/determining concentrations of light absorbing materials in blood comprising:
a measurement device main body determining concentrations of light absorbing materials in blood on the basis of a photoplethysmogram detected by irradiating and passing a plurality of optical signals of different wavelengths onto and through a living tissue;
simulating-signal generating means, for generating an arbitrary simulating-pulse-wave signal corresponding to the photoplethysmogram detected by a probe, provided in the measurement device main body
a bypass interconnection, arranged in the measurement device main body, routed so as to bypass a probe to be adapted; and
signal switching means for selectively inputting the photoplethysmogram detected by the probe and the simulating-pulse-wave signal which is transmitted through the bypass interconnection into a signal input section of the measurement device main body.

7. The apparatus for measuring/determining concentrations of light absorbing materials in blood according to claim 6, wherein the simulating-pulse-wave signal produced by the simulating signal generating means identifies an anomalous condition of the measurement device main body as a self checkup function in such a manner that the signal switching means selectively inputs, to the signal input section, a signal transmitted through the bypass interconnection and a signal which is detected as a simulating photoplethysmogram by receiving the optical signals having been irradiated corresponding to the simulating-pulse-wave signal in the probe.

8. The apparatus for measuring/determining concentrations of light absorbing materials in blood according to claim 6, wherein the simulating-pulse-wave signal produced by the simulating signal generating means identifies a normal operating state of the apparatus, an anomalous condition of the probe, and an anomalous condition of the measurement device main body, as a self checkup function in such a manner that the signal switching means selectively inputs, to the signal input section, a signal transmitted through the bypass interconnection and a received-light signal corresponding to the simulating-pulse-wave signal which is detected as a result of the optical signals having been irradiated in the probe.

9. The apparatus for measuring/determining concentrations of light absorbing materials in blood according to claim 8, further comprising:
a display section for displaying an checkup status of the apparatus, a normal operating status of the apparatus, an anomalous condition of the probe, and an anomalous condition of the measurement device main body.

10. The apparatus for measuring/determining concentrations of light absorbing materials in blood according to claim 6 further comprising:
signal conversion means, for converting the simulating-pulse-wave signal into a photoplethysmogram signal detected by the probe, provided with the bypass interconnection.

11. The apparatus for measuring/determining concentrations of light absorbing materials in blood according to claim 6, wherein the simulating signal generating means controls a time period for irradiating respective light-emitting diodes in accordance with the simulating-pulse-wave signal through pulse-width modulation (PWM) control to obtain a required simulating-pulse-wave received-light signal.

12. The apparatus for measuring/determining concentrations of light absorbing materials in blood according to claim 11, wherein the shape of a simulating pulse wave is set in accordance with a pulse-width modulation (PWM) pattern and in connection with a relationship between the pulse-width modulation (PWM) and a simulating-pulse-wave received-light signal.

13. The apparatus for measuring/determining concentrations of light absorbing materials in blood according to claim 11, wherein a pulsation component rate (the ratio between an AC component and a DC component) of a simulating pulse wave is set in accordance with a pulse-width modulation (PWM) ratio and in connection with a relationship between the pulse-width modulation (PWM) and a simulating-pulse-wave received-light signal.

14. The apparatus for measuring/determining concentrations of light absorbing materials in blood according to claim 11, wherein a simulating pulse rate is set in accordance with a modulation cycle and in connection with a relationship between the pulse-width modulation and a simulating-pulse-wave (PWM) received-light signal.

15. The apparatus for measuring/determining concentrations of light absorbing materials in blood according to claim 11, wherein a parameter pertaining to an absorption coefficient rate is set on the basis of a modulation ratio proportion between wavelengths and in connection with a relationship between the pulse-width modulation (PWM) and a simulating-pulse-wave received-light signal.

16. The apparatus for measuring/determining concentrations of light absorbing materials in blood according to claim 11, wherein the simulating-pulse-wave signal is formed from integral values obtained by integrating separated light-receiving time by a demodulation circuit section to each wavelength.

17. The apparatus for measuring/determining concentrations of light absorbing materials in blood according to claim 6, wherein the simulating signal generating means controls an extraction time required for demodulating a received-light signal stemming from emitting of respective light-emitting diodes being irradiated in accordance with the simulating-pulse-wave signal through pulse width modulation control to produce a required simulating-pulse-wave (PWM)received-light signal.

18. The apparatus for measuring/determining concentrations of light absorbing materials in blood according to claim 17, wherein the shape of a simulating pulse wave is set in accordance with a pulse-width modulation (PWM) pattern and in connection with a relationship between the pulse-width modulation (PWM) and a simulating-pulse-wave received-light signal.

19. The apparatus for measuring/determining concentrations of light absorbing materials in blood according to claim 17, wherein a pulsation component rate (the ratio between an AC component and a DC component) of a simulating pulse wave is set in accordance with a pulse-width modulation (PWM) ratio and in connection with a relationship between the pulse-width modulation (PWM) and a simulating-pulse-wave received-light signal.

20. The apparatus for measuring/determining concentrations of light absorbing materials in blood according to claim 17, wherein a simulating pulse rate is set in accordance with a modulation cycle and in connection with a relationship between the pulse-width modulation (PWM) and a simulating-pulse-wave received-light signal.

21. The apparatus for measuring/determining concentrations of light absorbing materials in blood according to claim 17, wherein a parameter pertaining to an absorption coefficient rate is set on the basis of a modulation ratio proportion between wavelengths and in connection with a relationship between the pulse-width modulation (PWM) and a simulating-pulse-wave received-light signal.

22. The apparatus for measuring/determining concentrations of light absorbing materials in blood according to claim 17, wherein the simulating-pulse-wave signal is formed from integral values obtained by integrating separated light-receiving time by a demodulation circuit section to each wavelength.
